(19) United States
(12) Patent Application Publication
(10) Pub. No.: US 2007/0027504 A1
(43) Pub. Date: Feb. 1, 2007

(54) Cranial nerve stimulation to treat a hearing disorder

(75) Inventors: Burke T. Barrett, Franklin, MA (US); Steven M. Parnis, Pearland, TX (US); Steven E. Maschino, Seabrook, TX (US); Albert W. Guzman, League City, TX (US)

Correspondence Address: CYBERONICS, INC., LEGAL DEPARTMENT, 6TH FLOOR, 100 CYBERONICS BOULEVARD, HOUSTON, TX, 77058, US

(73) Assignee: CYBERONICS, INC.

(21) Appl. No.: 11/190,796

(22) Filed: Jul. 27, 2005

(51) Int. Cl. A61N 1/00

(52) U.S. Cl. 607/55

(57) ABSTRACT

We disclose a method of treating a patient having a hearing disorder, including coupling at least one electrode to at least one vagus nerve of the patient and applying an electrical signal to the vagus nerve using the electrode to treat the hearing disorder. We also disclose a computer readable program storage device encoded with instructions that, when executed by a computer, perform the method, and a medical device and a hearing disorder treatment system that may be used in performance of the method.

### BACKGROUND OF THE INVENTION

The present invention relates generally to methods and apparatus for treating disorders by using cranial nerve stimulation. More particularly, it concerns methods and apparatus for treating hearing disorders by using vagus nerve stimulation.

There have been many improvements over the last several decades in medical treatments for disorders of the nervous system, such as epilepsy and other motor disorders, and abnormal neural discharge disorders. One of the more recently available treatments involves the application of an electrical signal to reduce various symptoms or effects caused by such neural disorders. For example, electrical signals have been successfully applied at strategic locations in the human body to provide various benefits, including reducing occurrences of seizures and/or improving or ameliorating other conditions. A particular example of such a treatment regimen involves applying an electrical signal to the vagus nerve of the human body to reduce or eliminate epileptic seizures, as described in U.S. Pat. No. 4,702,254 to Jacob Zabara, which is hereby incorporated in its entirety herein by reference in this specification. Electrical stimulation of the vagus nerve (hereinafter referred to as vagus nerve stimulation therapy or VNS) may be provided by implanting an electrical device underneath the skin of a patient and performing an electrical stimulation process, which may optionally include a sensor to detect a symptom of a disorder or condition of interest, which is used to trigger the electrical stimulation. Alternatively, the system may operate without a detection system once the patient has been diagnosed with a disorder, and may periodically apply a series of electrical pulses to the vagus (or other cranial) nerve intermittently throughout the day, or over another predetermined time interval.

A nerve bundle to which neurostimulation therapy is applied may comprise up to 100,000 or more individual nerve fibers of different types, including larger diameter A and B fibers which comprise a myelin sheath, and C fibers which have a much smaller diameter and are unmyelinated. Different types of nerve fibers respond differently to different types of stimulation signals. These different responses among nerve fiber types reflect, among other things, their different sizes, conduction velocities, stimulation thresholds, and myelination status (i.e., myelinated or unmyelinated). Therefore, the patient's body may respond differently depending on which type(s) of nerve fibers are the target of the stimulation therapy. In general, the larger, myelinated A and B fibers have a lower stimulation threshold than the unmyelinated, smaller C fibers.

A number of hearing disorders are known. One such hearing disorder is tinnitus, in which a patient perceives a sound when no external sound is present. Though colloquially known as ringing in the ears, a patient suffering tinnitus may perceive chirping, whistling, roaring, clicking, or other sounds. The American Tinnitus Association estimates about 50 million Americans suffer at least some degree of tinnitus, with about 12 million Americans suffering tinnitus severe enough to seek medical help and about 2 million Americans suffering debilitating tinnitus.

Auditory information is transmitted from the ear to the brain by afferent fibers of the vestibulocochlear nerve (eighth cranial nerve). However, a patient's particular case of a hearing disorder, such as tinnitus, may not be primarily caused by derangement of normal afferent signal transmission in the vestibulocochlear nerve. Known causes of tinnitus include jaw misalignment, cardiovascular disease, cranial nerve tumors, and head or neck trauma. Not to be bound by theory, these causes of tinnitus may alter the disposition of structures around the vestibulocochlear nerve, leading to impingement thereon. It is known that cranial nerves other than the vestibulocochlear nerve, such as the vagus nerve, innervate the portion of the head where the ear is located. The auricular branch of the vagus nerve innervates the skin of the back of the auricle (the flesh of the outer ear) and the posterior portion of the external acoustic meatus (the ear canal leading inward to the eardrum).

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 illustrates a neurostimulator system for stimulating the vagus nerve 100 of a patient, in accordance with one embodiment of the present invention.

FIG. 2 is a schematic rear view of a transverse cross-section of the head and neck of a person with attention to the vagus nerves and the auricular branches thereof, in accordance with one embodiment of the present invention.

FIG. 3 is a stylistic depiction of a close-up view of the left auricle 220l and nearby structures shown in FIG. 2.

FIG. 4 shows an exemplary electrical signal of a firing neuron as a graph of voltage at a given location at particular times during firing, in accordance with one embodiment of the present invention.

FIGS. 5A-5B show block diagrams of medical devices, in accordance with one embodiment of the present invention.

FIG. 6 shows a flowchart of a method in accordance with one embodiment of the present invention.

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed, but on the contrary, the intention is to cover all modifications, equivalents, and alternatives falling within the spirit and scope of the invention as defined by the appended claims.

### DETAILED DESCRIPTION

Certain terms are used throughout the following description and claims to refer to particular system components. As one skilled in the art will appreciate, components may be referred to by different names. This document does not intend to distinguish between components that differ in name but not function. In the following discussion and in the claims, the terms “including” and “comprising” are used in an open-ended fashion, and thus should be interpreted to mean “including, but not limited to.” Also, the term “couple” or “couples” is intended to mean either a direct or an indirect electrical connection. For example, if a first device couples to a second device, that connection may be through a direct electrical connection or through an indirect electrical connection via other devices, biological tissues, or magnetic fields. “Direct contact,” “direct attachment,” or providing a “direct coupling” indicates that a surface of a first element contacts the surface of a second element with no substantial attenuating medium therebetween. The presence of substances, such as bodily fluids, that do not substantially attenuate electrical connections does not vitiate direct contact. The word “or” is used in the inclusive sense (i.e., “and/or”) unless a specific use to the contrary is explicitly stated. All patents and patent applications specifically referred to herein are hereby incorporated by reference in the present application.

Illustrative embodiments of the invention are described herein. In the interest of clarity, not all features of an actual implementation are described in this specification. In the development of any such actual embodiment, numerous implementation-specific decisions must be made to achieve the design-specific goals, which will vary from one implementation to another. It will be appreciated that such a development effort, while possibly complex and time-consuming, would nevertheless be a routine undertaking for persons of ordinary skill in the art having the benefit of this disclosure.

Embodiments of the present invention may provide for the treatment of hearing disorders, such as tinnitus, by stimulating a cranial nerve, such as the vagus nerve.

Cranial nerve stimulation has been used successfully to treat a number of nervous system disorders, including epilepsy and other movement disorders, depression and other neuropsychiatric disorders, dementia, coma, migraine headache, obesity, eating disorders, sleep disorders, cardiac disorders (such as congestive heart failure and atrial fibrillation), hypertension, endocrine disorders (such as diabetes and hypoglycemia), and pain, among others. See, e.g., U.S. Pats. Nos. 4,867,164; 5,299,569; 5,269,303; 5,571,150; 5,215,086; 5,188,104; 5,263,480; 6,587,719; 6,609,025; 5,335,657; 6,622,041; 5,916,239; 5,707,400; 5,231,988; and 5,330,515. Despite the recognition that cranial nerve stimulation may be an appropriate treatment for the foregoing conditions, the fact that detailed neural pathways for many (if not all) cranial nerves remain relatively unknown makes predictions of efficacy for any given disorder difficult. Even if such pathways were known, moreover, the precise stimulation parameters that would energize particular pathways that affect the particular disorder likewise may be difficult to predict.

Accordingly, cranial nerve stimulation, and particularly vagus nerve stimulation, has not heretofore been deemed appropriate or effective for use in treating hearing disorders.

Disclosed herein is a method for treating a hearing disorder using stimulation of the vagus nerve (also known as the tenth cranial nerve). One or more other cranial nerves may be stimulated in addition to the vagus nerve, including the trigeminal nerve (as the fifth cranial nerve), the vestibulocochlear nerve (eighth cranial nerve), and the glossopharyngeal nerve (ninth cranial nerve), among others. A generally suitable form of neurostimulator for use in the method and apparatus of the present invention is disclosed, for example, in U.S. Pat. No. 5,154,172, assigned to the same assignee as the present application. The neurostimulator may be referred to as a NeuroCyb emetic Prosthesis (NCP®, Cyberonics, Inc., Houston, Tex., the assignee of the present application).

Certain parameters of the electrical stimuli generated by the neurostimulator are programmable. Programming the neurostimulator may be performed in a variety of manners, including those known to persons skilled in the art having benefit of the present disclosure

In one embodiment, the present invention relates to a method of treating a patient having a hearing disorder including coupling at least one electrode to at least one vagus nerve of the patient and applying an electrical signal to the vagus nerve using the electrode to treat the hearing disorder.

As used herein, the term “at least one vagus nerve” refers to the group consisting of the left vagus nerve and the right vagus nerve. The term “vagus nerve” may refer to a portion of the main trunk or a branch of a vagus nerve or plexus including vagus nerve fibers. In one embodiment, the invention comprises coupling the electrode to a left or right vagus nerve in the neck area of the patient's body. In alternative embodiments, the invention comprises coupling the electrode to a left or right vagus nerve in a near-diaphragmatic location, which may comprise either a sub-diaphragmatic location or a supra-diaphragmatic location. In another embodiment, the coupling of the electrode may include coupling the electrode to an auricular branch of the vagus nerve. In a further embodiment, the coupling of the electrode to the auricular branch may include positioning the electrode on the skin of the patient. The electrode may be positioned on the skin of the upper posterior of the auricle.

In one embodiment, treating the hearing disorder may include treating tinnitus.

Applying the electrical signal to the vagus nerve may include generating a response selected from the group consisting of an afferent action potential, an efferent action potential, an afferent hyperpolarization, and an efferent hyperpolarization. In one embodiment, the applying the electrical signal to the vagus nerve may include generating an afferent action potential. In one embodiment, the method may further include providing a programmable electrical signal generator, coupling the at least one electrode to the signal generator, generating an electrical signal with the electrical signal generator, and applying the electrical signal to the electrode.

In one embodiment, the method may further include programming the electrical signal generator to define the electrical signal by at least one parameter selected from the group consisting of a current magnitude, a pulse frequency, and a pulse width, wherein the parameter is selected to treat the hearing disorder.

In one embodiment, the method may further include detecting a symptom of the hearing disorder, wherein applying the electrical signal to the vagus nerve is initiated in response to the step of detecting the symptom of the hearing disorder. In a further embodiment, detecting the symptom of the hearing disorder may be performed by the patient. This may involve a subjective observation by the patient that he is experiencing a symptom of the hearing disorder. Alternatively or in addition, the symptom may be detected by performing a hearing test on the patient, as tinnitus or other hearing disorders typically interfere with a patient's ability to fully hear, or by visualizing brain function by an EKG, MRI, or PET scan to observe any cortical response typical of the hearing disorder.

The method may be performed under a single treatment regimen or under multiple treatment regimens. “Treatment regimen” herein refers to a parameter of the electrical signal, a duration for applying the signal, or a duty cycle of the signal, among others. In one embodiment, applying the electrical signal to the vagus nerve is performed during a first treatment period, and the method further includes applying a second electrical signal to the vagus nerve using the electrode during a second treatment period. In a further embodiment, the method may further include detecting a symptom of the hearing disorder, wherein the detecting the symptom is performed by the patient and the second treatment period is initiated in response to the patient detecting a symptom of the hearing disorder. For example, a patient suffering a hearing disorder typically presenting with a set of chronic symptoms, but who also periodically suffers acute episodes of the hearing disorder presenting a set of symptoms that is different from or more intense than one or more chronic symptoms, may benefit by receiving a first electrical signal during a first, chronic treatment period and a second electrical signal during a second, acute treatment period. Three or more treatment periods may be used, if deemed desirable by a medical practitioner.

In treating the hearing disorder, in certain embodiments the electrode may be directly coupled to the vagus nerve, e.g., by providing a surgical attachment.

In one particular embodiment, the present invention relates to a method of treating a patient having a hearing disorder, including coupling at least one electrode to at least one vagus nerve of the patient, providing an electrical signal generator coupled to the at least one electrode, generating an electrical signal with the electrical signal generator, and applying the electrical signal to the electrode to treat the hearing disorder. The invention may further comprise detecting a symptom of the hearing disorder, wherein applying the electrical signal to the vagus nerve is initiated in response to detecting the symptom. The method may comprise coupling the electrode to an auricular branch of the vagus nerve.

In another embodiment, the invention comprises a method of treating a patient with a hearing disorder by coupling at least one electrode to an auricular branch of the vagus nerve of the patient, and applying an electrical signal to the auricular branch of the vagus nerve using the electrode. The method may further comprise providing a programmable electrical signal generator, coupling the at least one electrode to the signal generator, generating an electrical signal with the electrical signal generator, and applying the electrical signal to the auricular branch may comprise applying the electrical signal to the at least one electrode. The invention may further comprise programming the electrical signal generator to define the electrical signal by a plurality of parameters selected from the group consisting of a current magnitude, a pulse frequency, a pulse width, an on-time and an off-time. In another embodiment, the step of applying en electrical signal to the auricular branch includes applying the signal during a first treatment period, and the method further comprises applying a second electrical signal to the auricular branch during a second treatment period. The first treatment period may comprise a period ranging from one hour to six months, and the second treatment period may comprise a period ranging from one month to the patient's lifetime.

In one embodiment, the present invention relates to a computer readable program storage device encoded with instructions that, when executed by a computer, perform a method including generating an electrical signal and providing the electrical signal to a vagus nerve of a patient by using an electrode to treat a hearing disorder.

In one embodiment wherein the computer readable program storage device encoded with instructions that, when executed by a computer, performs the method, the electrical signal may be a controlled current electrical signal.

In one embodiment wherein the computer readable program storage device encoded with instructions that, when executed by a computer, performs the method, the method may further include programming an electrical signal generator to define the electrical signal by at least one parameter selected from the group consisting of a current magnitude, a pulse frequency, and a signal width, wherein the parameter is selected to treat the hearing disorder.

In one embodiment wherein the computer readable program storage device encoded with instructions that, when executed by a computer, performs the method, the method may further include detecting a symptom of the hearing disorder, wherein providing the electrical signal is initiated in response to detecting the symptom.

In one embodiment, the present invention relates to a hearing disorder treatment system, including at least one electrode coupled to at least one vagus nerve of a patient and an implantable device operatively coupled to the electrode and including an electrical signal generator capable of applying an electrical signal to the vagus nerve using the electrode to treat the hearing disorder.

The at least one electrode and its coupling to the at least one vagus nerve may be as described above.

The electrical signal generator may be capable of triggering an afferent action potential. The electrical signal generator may be a programmable electrical signal generator. The electrical signal generator may be capable of defining the electrical signal by at least one parameter selected from the group consisting of a current magnitude, a pulse frequency, and a pulse width, wherein the at least one parameter is selected to treat the hearing disorder. The hearing disorder treatment system may further include a detection communicator capable of delivering, directly or indirectly, at least one signal to the electrical signal generator, and wherein the electrical signal generator is capable of applying the electrical signal on receipt of the at least one signal from the detection communicator. In a further embodiment, the at least one signal communicated by the detection communicator may be generated by the patient.

Specific embodiments of the present invention will now be discussed with reference to the various figures.

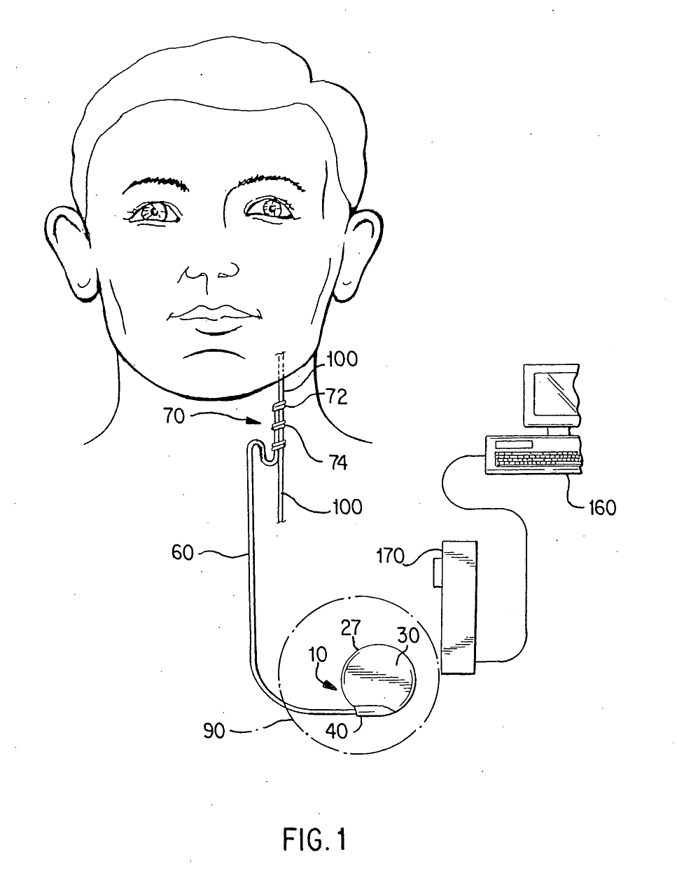

FIG. 1 illustrates a neurostimulator system for stimulating the vagus nerve 100 of a patient, in accordance with one embodiment of the present invention. Electrical signal generator 10 may be provided with a main body 30 including a case or shell 27 with a header 40 having one or more electrical connectors for connecting to leads 60. The generator 10 may be implanted in the patient's chest in a pocket or cavity formed by the implanting surgeon below the skin (indicated by dotted line 90), similar to the implantation procedure for a pacemaker pulse generator. A stimulating nerve electrode assembly 70, such as one including an electrode pair 72, 74, may be conductively connected to the distal end of an insulated electrically conductive lead assembly 60, which may include a pair of lead wires (one wire for each electrode of an electrode set). Each lead wire in lead assembly 60 may be attached at its proximal end to a connector 50 on case 27. The electrode assembly 70 may be surgically coupled to a vagus nerve 100 at a target location, such as the patient's neck as shown in FIG. 1. Alternatively, the electrode assembly may be coupled to the vagus nerve at a location near the diaphragm of the patient, which may include a supra- or sub-diapraghmatic location. In another embodiment, the electrode assembly may be coupled to the vagus nerve at the auricular branch of a vagus nerve 100.

The electrode assembly 70 may include a bipolar stimulating electrode pair, such as the electrode pair described in U.S. Pat. No. 4,573,481 to Bullara, Mar. 4, 1986. The skilled artisan having the benefit of the present disclosure may appreciate that many electrode designs may be used in the present invention. The electrodes preferably directly contact the vagus nerve 100. As shown in FIG. 1, in a particular embodiment, a spiral electrode may be wrapped about the vagus nerve 100, and the electrode assembly 70 may be secured to the vagus nerve 100 by a spiral anchoring tether, such as that disclosed in U.S. Pat. No. 4,979,511 to Terry, Jr., Dec. 25, 1990 and assigned to the same assignee as the present application. Lead assembly 60 may be secured while retaining the ability to flex with movement of the chest and neck by a suture connection to nearby tissue. While the electrodes 72, 74 of the electrode assembly 70 are shown in FIG. 1 directly contacting the vagus nerve 100, the skilled artisan having the benefit of the present disclosure may appreciate that embodiments in which the electrodes do not directly contact the nerve but are electrically coupled to it are possible.

Electrode assembly 70 may conform to the shape of the nerve, providing a low stimulation threshold by allowing a large stimulation contact area with the nerve. In one embodiment, the electrode assembly 70 may include two electrode ribbons (not shown), formed of a conductive material such as platinum, iridium, platinum-iridium alloys, or oxides of the foregoing. The electrode ribbons may be individually bonded to an inside surface of an elastomeric body portion of the spiral electrodes 72, 74.

Lead assembly 60 may include two distinct lead wires or a coaxial cable with two conductive elements respectively coupled to one of the conductive electrode ribbons 72, 74. One suitable method of coupling the lead wires or cable to the electrodes comprises a spacer assembly such as that disclosed in U.S. Pat. No. 5,531,778, although other coupling techniques may be used. The elastomeric body portion of each loop may be formed of silicone rubber. Although FIG. 1 illustrates a system for stimulating the left vagus nerve in the neck (cervical) area, the skilled artisan having the benefit of the present disclosure will understand the stimulation signal may be applied to the right cervical vagus nerve in addition to or instead of the left vagus nerve, and all such embodiments are within the scope of the present invention. In such embodiments, lead and electrode assemblies substantially as discussed above may be coupled to the same or a different generator. FIG. 1 also illustrates an external programming system capable of wireless (e.g., radio frequency, RF) communication with the signal generator 10, which may be used to program a therapeutic electrical signal in the signal generator. The external programming system may include a wand 170 having an RF transmitter and receiver, and a computer 160, which may include a handheld computer operable by a healthcare practitioner. Wand 170 may communicate with a receiver and transmitter in signal generator 10, and may be used to receive date from or transmit data to the signal generator 10. Other communications systems, such as communication systems without a wand and operating in the MICS band at 402-405 MHz, may also be used.

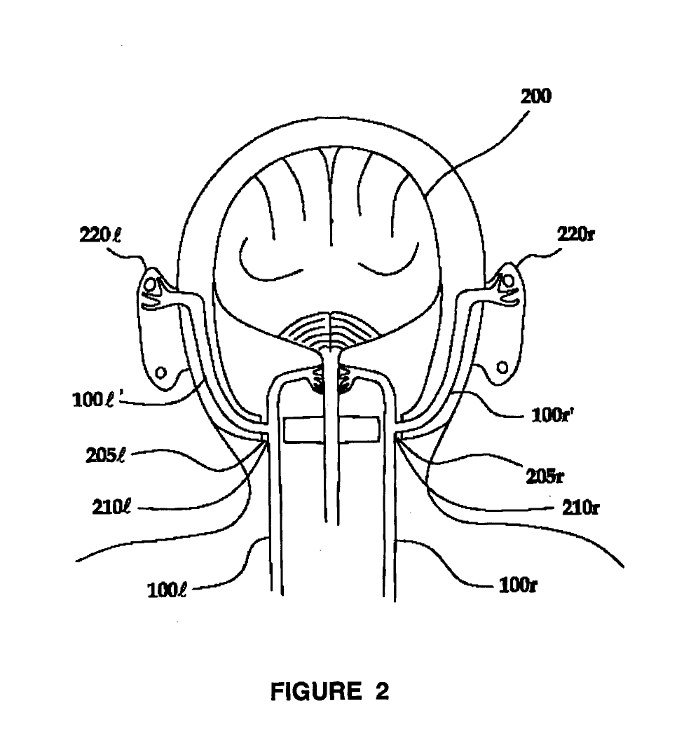

FIG. 2 is a schematic rear view of a transverse cross-section of the head and neck of a person with attention to the vagus nerves and the auricular branches thereof, in accordance with one embodiment of the present invention. The left and right vagus nerves 100l, 100r emerge from the brain 200 and exit the skull at the left and right jugular foramina 205l, 205r. At the foramina 205l, 205r are found superior ganglia 210l, 210r of the left and right vagus nerves 100l, 1000r, from which emerge left and right auricular branches 100l′, 100r′.

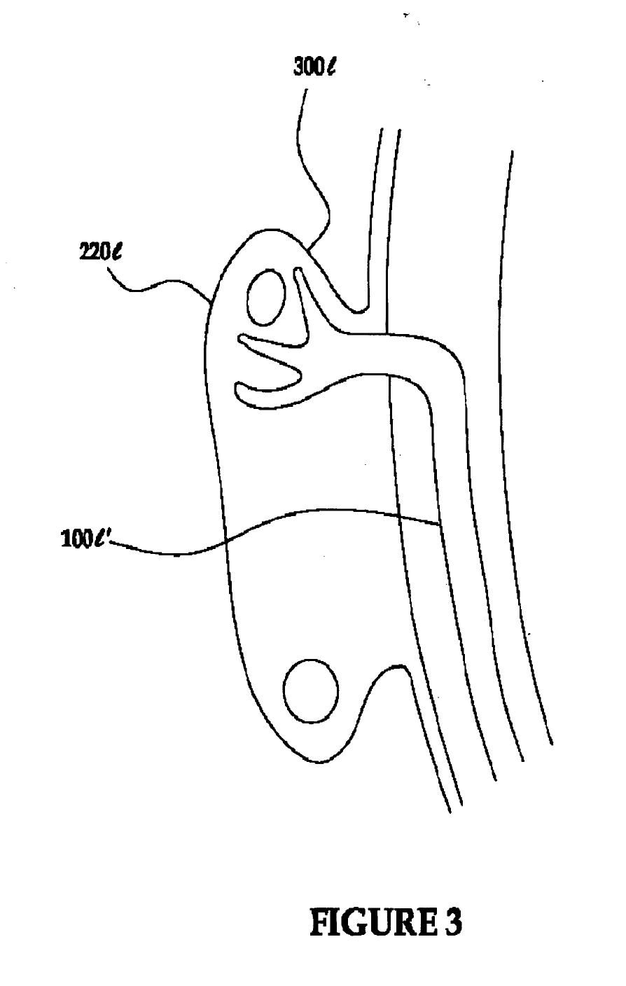

FIG. 3 is a schematic close-up view of the left auricle 220l and nearby structures shown in FIG. 2, in accordance with one embodiment of the present invention. The left auricular branch 1001′ innervates the auricle and lies relatively close to the skin of the back 300l of the auricle 220l. In one embodiment, a lead assembly and associated electrodes may be positioned on the skin of the back 300l of the left auricle 220l (or the skin of the back of the right auricle, or both, not shown).

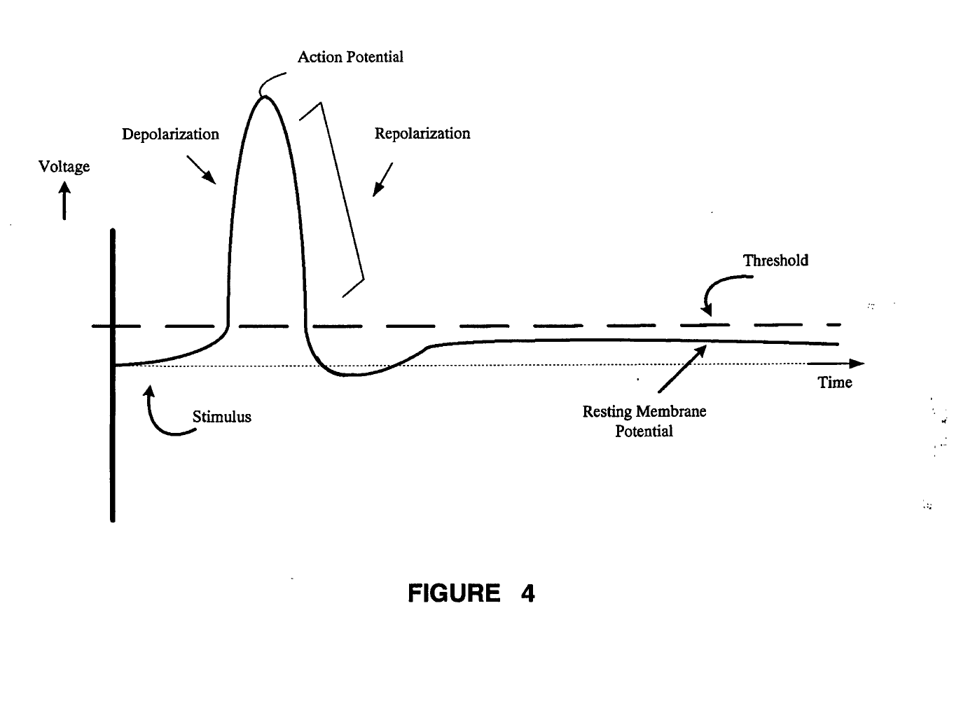

FIG. 4 shows an exemplary electrical signal of a firing neuron as a graph of voltage at a given location at particular times during firing, in accordance with one embodiment of the present invention. A typical neuron has a resting membrane potential of about −70 mV, maintained by transmembrane ion channel proteins. When a portion of the neuron reaches a firing threshold of about −55 mV, the ion channel proteins in the locality allow the rapid ingress of extracellular sodium ions, which depolarizes the membrane to about +30 mV. The wave of depolarization then propagates along the neuron. After depolarization at a given location, potassium ion channels open to allow intracellular potassium ions to exit the cell, lowering the membrane potential to about −80 mV (hyperpolarization). A depolarization interval is required for transmembrane proteins to return sodium and potassium ions to their starting intra- and extracellular concentrations and allow a subsequent action potential to occur. The present invention may raise or lower the resting membrane potential, thus making the reaching of the firing threshold more or less likely and subsequently increasing or decreasing the rate of fire of any particular neuron.

A cranial nerve may include afferent fibers, efferent fibers, or both. Afferent fibers transmit information to the brain from the extremities; efferent fibers transmit information from the brain to the extremities. The vagus nerve comprises both afferent and efferent fibers, and a neurostimulator may be used to stimulate both types of fibers.

A cranial nerve may include fibers that transmit information in the sympathetic nervous system, the parasympathetic nervous system, or both. Inducing an action potential in the sympathetic nervous system may yield a result similar to that produced by blocking an action potential in the parasympathetic nervous system and vice versa, but this is a general observation, not a rule seen in all cases.

Returning to FIG. 1, neurostimulator 10 may generate electrical signals according to one or more programmed parameters for stimulation of the vagus nerve 100. In one embodiment, the stimulation parameters may be selected from the group consisting of a current magnitude, a pulse frequency, a signal width, on-time, and off-time. A table of ranges for each of these stimulation parameters is provided in Table 1. The stimulation parameter may be of any suitable waveform known in the art of neurostimulation, e.g., a square wave. Various electrical signal patterns may be employed by the skilled artisan having the benefit of the present invention. These electrical signals may include a plurality of types of pulses, e.g., pulses with varying amplitudes, polarity, frequency, etc. Other types of signals may also be used, such as sinusoidal waveforms, etc. The electrical signal may be controlled current signals. In some embodiments, one or more of the parameters defining the electrical signal may comprise a random value within a defined range.

TABLE 1

| Parameter | Range |
|---|---|
| Output current | 0.1-6.0 mA |
| Pulse width | 10-1500 μsec |
| Frequency | 0.5-250 Hz |
| On-time | 1 sec and greater |
| Off-time | 0 sec and greater |
| Frequency Sweep | 10-100 Hz |
| Random Frequency | 10-100 Hz |

On-time and off-time parameters may be used to define an intermittent pattern in which a repeating series of signals is generated for stimulating the nerve during the on-time (such a sequence may be referred to as a “pulse burst”), followed by a period in which no signals are generated and the nerve is allowed to recover from the stimulation during the pulse burst. The on/off duty cycle of these alternating periods of stimulation and no stimulation may have a ratio in which the off-time may be set to zero, providing continuous stimulation, or it may be as long as one day or more, in which case the stimulation is provided once per day or at even longer intervals. Typically, however, the ratio off-time/on-time may range from about 0.5 to about 10.

Nominally, the width of each signal may be set to a value not greater than about 1 msec, such as about 250-500 μsec, and the signal repetition frequency may be programmed to be in a range of about 20-250 Hz. A nonuniform frequency may also be used. Frequency may be altered during a pulse burst by either a frequency sweep from a low frequency to a high frequency, or vice versa. Alternatively, the timing between adjacent individual signals within a burst may be randomly changed such that two adjacent signals may be generated at any frequency within a range of frequencies.

In one embodiment, the present invention may include coupling of at least one electrode to each of two or more cranial nerves. (In this context, two or more cranial nerves means two or more nerves having different names or numerical designations, and does not refer to e.g. the left and right versions of a particular nerve). In one embodiment, at least one electrode may be coupled to each of the vagus nerve and the vestibulocochlear nerve. Each of the nerves in this embodiment or others involving two or more cranial nerves may be stimulated according to particular activation modalities that may be independent between the two nerves.

Another activation modality for stimulation is to program the output of the neurostimulator to the maximum amplitude which the patient may tolerate, with cycling on and off for a predetermined period of time followed by a relatively long interval without stimulation. Where the cranial nerve stimulation system is completely external to the patient's body, higher current amplitudes may be needed to overcome the attenuation resulting from the absence of direct contact with the vagus nerve and the additional impedance of the skin of the patient. Although external systems typically require greater power consumption than implantable systems, they have an advantage in that their batteries may be replaced noninvasively.

External stimulation may be used as a screening test to determine if the patient should receive an implanted cranial nerve stimulation system. In one embodiment, the invention comprises stimulating the trigeminal nerve, the glossopharyngeal nerve, or the auricular branch of the vagus nerve with a skin-mounted electrode to determine if the patient is responsive to cranial nerve stimulation for treating the hearing disorder. In one embodiment, an electrode may be coupled to the skin of the back of the patient's auricle to stimulate the auricular branch of the vagus nerve. A lead may connect the skin electrode to an electrical pulse generator carried by the patient, e.g., in a pocket or mounted on a belt. The patient may be subjected to relatively high stimulation for a first test period to determine whether the patient's hearing disorder is amenable to treatment with cranial nerve stimulation.

In one embodiment, the symptoms of the patient may be analyzed following the first test period, and a decision may be made whether or not implantation of an implantable system is desirable. If the hearing disorder is treated, the patient may be considered for an implanted system providing direct coupling to a cranial nerve. In certain embodiments, both external stimulation and internal stimulation may be employed to treat the hearing disorder.

Other types of indirect stimulation may be performed in certain embodiments of the invention. In one embodiment, the invention comprises providing noninvasive transcranial magnetic stimulation (TMS) to the brain of the patient to treat the hearing disorder. TMS systems include those disclosed in U.S. Pats. Nos. 5,769,778; 6,132,361; and 6,425,852. Where TMS is used, it may be used in conjunction with cranial nerve stimulation as an adjunctive therapy. In some embodiments, TMS alone may be used to treat the hearing disorder. In one embodiment, both TMS and direct vagus nerve stimulation may be performed to treat the hearing disorder.

Returning to systems for providing direct cranial nerve stimulation, such as that shown in FIG. 1, stimulation may be provided in at least two different modalities. Where cranial nerve stimulation is provided based solely on programmed off-times and on-times, the stimulation may be referred to as passive, inactive, or non-feedback stimulation. In contrast, stimulation may be triggered by one or more feedback loops according to changes in the body or mind of the patient. This stimulation may be referred to as active or feedback-loop stimulation. In one embodiment, feedback-loop stimulation may be manually triggered stimulation, in which the patient manually causes the activation of a pulse burst outside of the programmed on-time/off-time cycle. For example, if the patient undergoes an acute episode of the hearing disorder, he may manually activate the neurostimulator to stimulate the cranial nerve to treat the acute episode. The patient may also be permitted to alter the intensity of the signals applied to the cranial nerve within limits established by the physician. For example, the patient may be permitted to alter the signal frequency, current, duty cycle, or a combination thereof. In at least some embodiments, the neurostimulator may be programmed to generate the stimulus for a relatively long period of time in response to manual activation.

Patient activation of a neurostimulator may involve use of an external control magnet for operating a reed switch in an implanted device, for example. Certain other techniques of manual and automatic activation of implantable medical devices are disclosed in U.S. Pat. No. 5,304,206 to Baker, Jr., et al., assigned to the same assignee as the present application (“the '206 patent”). According to the '206 patent, means for manually activating or deactivating a stimulus generator may include a sensor such as piezoelectric element mounted to the inner surface of the generator case and adapted to detect light taps by the patient on the implant site. One or more taps applied in fast sequence to the skin above the location of the stimulus generator in the patient's body may be programmed into the device as a signal for activation of the generator, whereas two taps spaced apart by a slightly longer duration of time may be programmed into the device as a signal for deactivation of the generator, for example. The therapy regimen performed by the implanted device may remain that which has been preprogrammed by means of an external programmer, according to the prescription of the patient's physician in concert with recommended programming techniques provided by the device manufacturer. In this way, the patient may be given limited but convenient control over operation of the device to an extent which may be determined by the program dictated or entered by the attending physician. The patient may also activate the neurostimulator using other suitable techniques or apparatus.

In some embodiments, feedback stimulation systems other than manually-initiated stimulation may be used in the present invention. A cranial nerve stimulation system may include a sensing lead coupled at its proximal end to a header along with a stimulation lead and electrode assemblies. A sensor may be coupled to the distal end of the sensing lead. The sensor may include a temperature sensor, a blood parameter sensor, a heart parameter sensor, a brain parameter sensor, or a sensor for another body parameter. The sensor may also include a nerve sensor for sensing activity on a nerve, such as a cranial nerve, such as the vagus nerve or the vestibulocochlear nerve. In one embodiment, the sensor may sense a body parameter that corresponds to a symptom of the hearing disorder. If the sensor is to be used to detect a symptom of the hearing disorder, a signal analysis circuit may be incorporated into the neurostimulator for processing and analyzing signals from the sensor. Upon detection of the symptom of the hearing disorder, the processed digital signal may be supplied to a microprocessor in the neurostimulator device to trigger application of the stimulating signal to the cranial nerve. In another embodiment, the detection of a symptom of interest may trigger a stimulation program including different stimulation parameters from a passive stimulation program, such as having a higher current or a higher ratio of on-time to off-time.

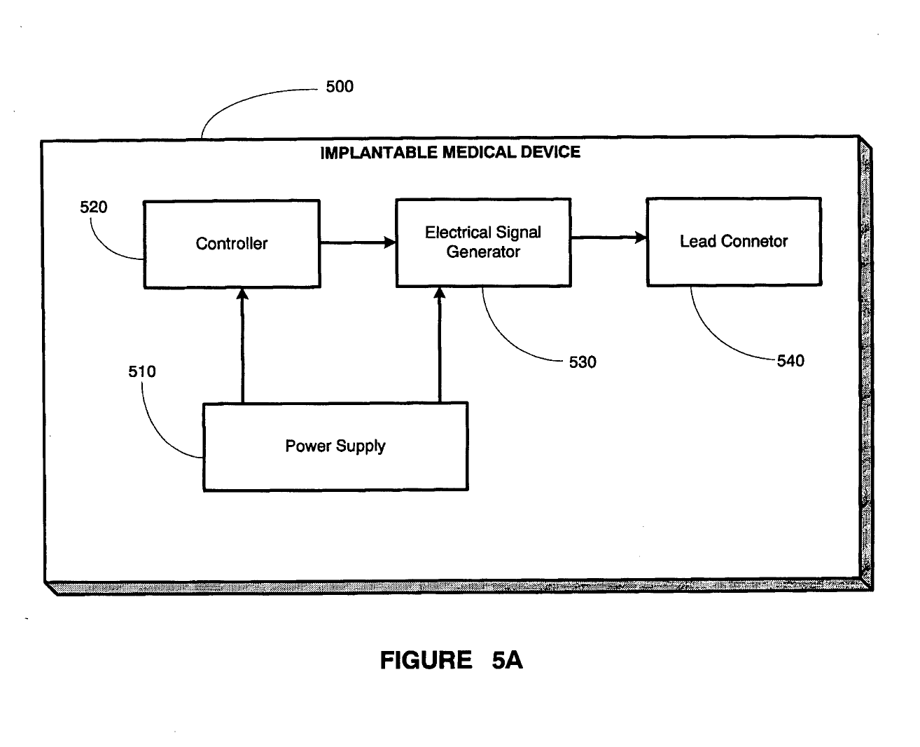

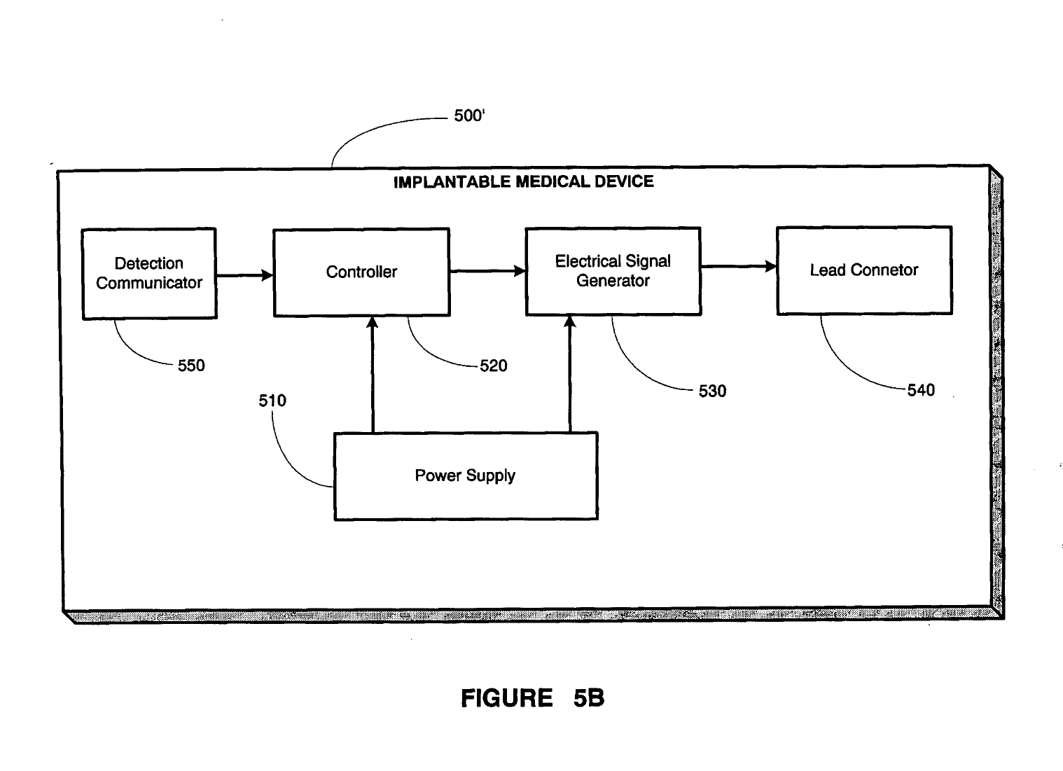

FIG. 5A shows a block diagram depiction of a medical device 500, in accordance with one embodiment of the present invention. The medical device 500 comprises a power supply 510 capable of providing power to an operation performed by the medical device; a controller 520 to authorize generation of an electrical signal, and an electrical signal generator 530 to generate an electrical signal upon authorization by the controller and providing the electrical signal to a lead connector 640. FIG. 5B shows a block diagram of an alternative medical device 500′, in accordance with one embodiment of the present invention, including the power supply 510, controller 520, electrical signal generator 530, and lead connector 540 referred to above, and further including a further including a detection communicator 550, wherein the power supply 510 is capable of providing power to the detection communicator 550, the detection communicator 550 is capable of delivering at least one signal to the controller 520, and the controller 520 is capable upon receipt of the at least one signal from the detection communicator 550 of authorization of generating an electrical signal by the electrical signal generator 530.

In one embodiment, the controller 520 defines stimulation pulses to be delivered to the nerve tissue according to parameters that may be preprogrammed into the device 500. The controller 520, which may include a processor that can execute program code, controls the operation of the electrical signal generator 530, which generates the stimulation pulses according to programmed parameters and provides these pulses to the lead connector 540 for delivery to the patient. The controller 520 may be capable of implementing multi-phasic controlled current signal outputs. The controller 520 may be capable of providing a controlled current signal where pulses may comprise various amplitudes, varying phases, and varying polarity. The controller 520 may also be capable of providing mono-phasic stimulation signals. The controller 520 may also be capable of switching between various electrodes employed by the device 500.

In an alternative embodiment, based upon various parameters provided to the device 500, the controller 520 may develop a multi-phasic pulse description pattern and provide the same to the electrical signal generator 530 to perform a particular type of multi-phasic stimulation. The controller 520 may be capable of converting stored data relating to the phasic pulse description and may control behavior of the electrical signal generator 530 accordingly. Additionally, the device 500 also may include a burst description array that comprises data relating to performing a pulse-to-pulse variation of a stimulation signal. The controller 520 may be capable of using data from the burst description array to provide a stimulation signal that comprises a pulse train, where one pulse in the pulse train may vary from another pulse train. This pulse-to-pulse variation may include variations in the pulse width, amplitude, pulse-shape, polarity, etc.

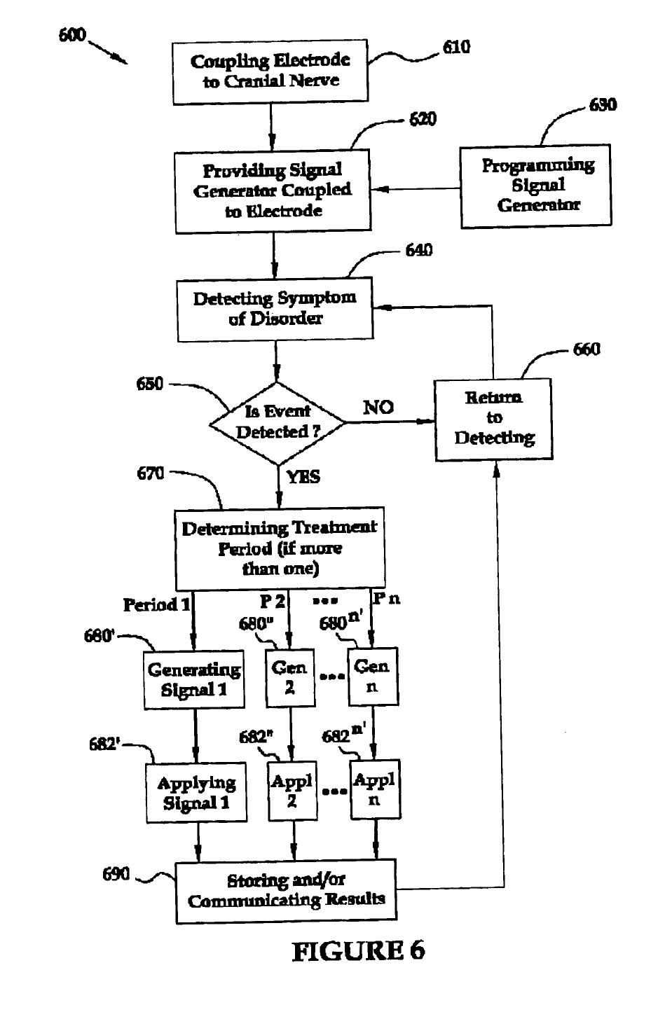

FIG. 6 provides a flowchart of the steps of a method 600 in accordance with one embodiment of the present invention. Method 600 comprises coupling 610 at least one electrode to at least one cranial nerve of a patient and providing 620 a signal generator coupled to the electrode. The signal generator may programmed in a programming step 630. After the electrode has been coupled 610 and the signal generator has been provided 620, the method 600 may include detecting 640 an event indicative of a symptom of a disorder to be treated. At each execution 650 of the detecting step 640, if an event is not detected, the flow of the method 600 returns 660 to detecting 640. If an event is detected during execution 650, the flow of the method 600 moves to determining 670 the treatment period to implement, if more than one is intended by the healthcare practitioner implementing the method 600. FIG. 6 shows a number n of treatment periods designated prime, double prime . . . , n-prime. Each treatment period comprises generating 680′, 680″ . . . , 680^{n′} a signal and applying 682′, 682″ . . . , 682n′ the signal to the electrode coupled 610 to the cranial nerve. After treatment, the results of the treatment may be stored or communicated to other steps in the method 600, such as returning 660 to the detecting step 640.

All of the methods and apparatus disclosed and claimed herein may be made and executed without undue experimentation in light of the present disclosure. While the methods and apparatus of this invention have been described in terms of particular embodiments, it will be apparent to those of skill in the art that variations may be applied to the methods and apparatus and in the steps or in the sequence of steps of the method described herein without departing from the concept, spirit and scope of the invention as defined by the appended claims. It should be especially apparent that the principles of the invention may be applied to selected cranial nerves other than the vagus nerve to achieve particular results.

What is claimed is:

1. A method of treating a patient having a hearing disorder, comprising:
coupling at least one electrode to a vagus nerve of the patient, and
applying an electrical signal to the vagus nerve using the electrode to treat the hearing disorder.

2. The method of claim 1, wherein the hearing disorder is tinnitus.

3. The method of claim 1, wherein coupling at least one electrode comprises coupling the electrode to an auricular branch of the vagus nerve.

4. The method of claim 3, wherein coupling at least one electrode comprises positioning the electrode on the skin of the patient.

5. The method of claim 1, wherein applying an electrical signal to the vagus nerve comprises generating a response selected from the group consisting of an afferent action potential, an efferent action potential, an afferent hyperpolarization, and an efferent hyperpolarization.

6. The method of claim 1, further comprising generating an afferent action potential on said vagus nerve using said electrical signal.

7. The method of claim 1, further comprising the steps of:
providing a programmable electrical signal generator;
coupling said at least one electrode to said signal generator;
generating an electrical signal with the electrical signal generator;
and wherein applying an electrical signal to the vagus nerve comprises applying the electrical signal to the electrode.

8. The method of claim 7, further comprising:
programming the electrical signal generator to define said electrical signal by at least one parameter selected from the group consisting of a current magnitude, a pulse frequency, and a pulse width, wherein electrical signal is adapted to treat the hearing disorder.

9. The method of claim 1, further comprising detecting a symptom of the hearing disorder, wherein applying an electrical signal to a vagus nerve is initiated in response to said step of detecting a symptom of the hearing disorder.

10. The method of claim 10, wherein detecting a symptom of the hearing disorder is performed by the patient.

11. The method of claim 1, wherein applying an electrical signal to the vagus nerve comprises applying said signal during a first treatment period, and said method further comprises applying a second electrical signal to the vagus nerve during a second treatment period.

12. The method of claim 11, further comprising detecting a symptom of the hearing disorder, wherein said detecting step is performed by the patient;
and wherein applying said second electrical signal is initiated in response to said step of detecting a symptom of the hearing disorder.

13. The method of claim 1 wherein coupling at least one electrode comprises contacting said at least one electrode directly to a vagus nerve.

14. A method of treating a patient having a hearing disorder, comprising:
coupling at least one electrode to a vagus nerve of the patient;
providing an electrical signal generator coupled to the at least one electrode;
generating an electrical signal with the electrical signal generator; and
applying the electrical signal to the electrode to treat the hearing disorder.

15. The method of claim 14, further comprising the step of detecting a symptom of the hearing disorder, wherein the step of applying the electrical signal to the vagus nerve is initiated in response to detecting said the symptom.

16. The method of claim 14 wherein coupling at least one electrode to a vagus nerve comprises coupling at least one electrode to an auricular branch of a vagus nerve.

17. A method of treating a patient having a hearing disorder, comprising:
coupling at least one electrode to an auricular branch of a vagus nerve of the patient, and
applying an electrical signal to said auricular branch of a vagus nerve using the electrode to treat the hearing disorder.

18. The method of claim 17 further comprising:
providing a programmable electrical signal generator;
coupling said at least one electrode to said signal generator;
generating an electrical signal with the electrical signal generator;
and wherein applying an electrical signal to said auricular branch comprises applying the electrical signal to said at least one electrode.

19. The method of claim 18, further comprising:
programming the electrical signal generator to define said electrical signal by a plurality of parameters selected from the group consisting of a current magnitude, a pulse frequency, a pulse width, an on-time and an off-time.

20. The method of claim 17, wherein applying an electrical signal to said auricular branch comprises applying said signal during a first treatment period, and said method further comprises applying a second electrical signal to said auricular branch of a vagus nerve during a second treatment period.

21. The method of claim 19, wherein said first treatment period comprises a period ranging from one hour to six months, and wherein said second treatment period comprises a period ranging from one month to the patient's lifetime.
